(19) United States
(12) Patent Application Publication
(10) Pub. No.: US 2023/0200747 A1
(43) Pub. Date: Jun. 29, 2023

(54) METHOD, SERVER, DEVICE, AND NON-TRANSITORY COMPUTER-READABLE RECORDING MEDIUM FOR MONITORING BIOSIGNALS USING WEARABLE DEVICE

(71) Applicant: HUINNO, CO., LTD., Seoul (KR)

(72) Inventors: Joo Min KIM, Seoul (KR); Yeong Joon GIL, Busan (KR); Sung Hoon JUNG, Busan (KR); Doo Seok PARK, Seoul (KR)

(21) Appl. No.: 17/927,174

(22) PCT Filed: Dec. 10, 2021
(86) PCT No.: PCT/KR2021/018749

§ 371 Date: Nov. 22, 2022

(30) Foreign Application Priority Data
Dec. 17, 2020 (KR) 10-2020-0177857

(51) Int. Cl. A61B 5/00

(52) U.S. Cl. CPC A61B 5/7282; A61B 5/7203; A61B 5/7225; A61B 5/0022; A61B 5/6802

(57) ABSTRACT

A method for monitoring biosignals using a wearable device is provided. The method includes the steps of: acquiring information on a result of performing a primary analysis of a biosignal measured by the device, using a primary analysis model trained to perform a primary analysis for detecting abnormal events from biosignals, and acquiring, from the biosignal, a partial biosignal associated with the result of performing the primary analysis; and performing a secondary analysis of the partial biosignal with reference to the information on the result of performing the primary analysis, using a secondary analysis model trained to perform a secondary analysis for detecting abnormal events from biosignals, wherein the primary analysis model is a relatively light-weighted model compared to the secondary analysis model.

### CROSS-REFERENCE TO RELATED APPLICATIONS

This application is a National Stage Entry of International Application No. PCT/KR2021/018749 filed on Dec. 10, 2021, which claims priority from Korean Application No. 10-2020-0177857 filed on Dec. 17, 2020. The aforementioned applications are incorporated herein by reference in their entireties.

### TECHNICAL FIELD

The present invention relates to a method, server, device, and non-transitory computer-readable recording medium for monitoring biosignals using a wearable device.

### RELATED ART

In recent years, techniques have emerged that allow a user to easily and conveniently measure a biosignal such as an electrocardiogram at home without visiting a hospital, and diagnose a cardiac disorder such as arrhythmia on the basis of the measured biosignal.

As an example of related conventional techniques, Korean Laid-Open Patent Publication No. 2007-96620 discloses an electrocardiogram measurement apparatus, comprising: a biosignal measurement unit configured to measure a biosignal including an electrocardiogram; an electrocardiogram abnormality indication detection unit configured to detect an electrocardiogram abnormality indication by analyzing an electrocardiogram signal inputted from the biosignal measurement unit; a user activity state acquisition unit configured to acquire user activity state information when an abnormality indication detection signal is inputted from the electrocardiogram abnormality indication detection unit; an emergency situation determination unit configured to determine presence or absence of an electrocardiogram abnormality on the basis of the user activity state information inputted from the user activity state acquisition unit and the electrocardiogram signal inputted from the biosignal measurement unit; and an emergency situation notification unit configured to notify of the presence or absence of the electrocardiogram abnormality inputted from the emergency situation determination unit.

Further, in recent years, techniques have been introduced that constantly measure a biosignal using a wearable device constantly attached to a user's body, and accurately analyze the biosignal using an artificial intelligence algorithm such as machine learning. However, in order to involve an artificial intelligence algorithm in analyzing the biosignal, resources for processing a vast amount of data with high-level computing power are required. Thus, there is a limitation that it is difficult to implement an artificial intelligence algorithm in a wearable device composed of only limited resources.

In order to overcome this limitation, a technique has been introduced that implements an artificial intelligence-based analysis model in a remote server capable of wirelessly communicating with a wearable device. However, even with the conventional technique, there is still a limitation that a vast amount of biosignal data should be transmitted and received between the wearable device and the server, and it takes a considerable amount of time until an analysis result is derived by the artificial intelligence-based analysis model operating in the server, so that a considerable time difference occurs between time points of measurement and discrimination.

In this connection, the inventor(s) present a novel and inventive technique capable of accurately monitoring an abnormal event from a biosignal measured by a wearable device in real time using artificial intelligence-based analysis models, by performing a primary analysis of the biosignal using a light-weighted analysis model in the wearable device, and performing a secondary analysis of the biosignal with refence to a result of the primary analysis acquired from the wearable device, using an advanced analysis model in a server.

### DISCLOSURE

### Technical Problem

One object of the present invention is to solve all the above-described problems in the prior art.

Another object of the invention is to accurately monitor abnormal events from biosignals in real time using artificial intelligence-based analysis models and a wearable device, by performing a primary analysis of a biosignal using a light-weighted analysis model in the wearable device, and performing a secondary analysis of the biosignal with refence to a result of the primary analysis acquired from the wearable device, using an advanced analysis model in a server.

Yet another object of the invention is to reduce the data size of a biosignal measured by a wearable device while ensuring high quality of the biosignal, by removing low-frequency noise from the biosignal to reduce the number of bits of data extracted (or sampled) from an analog signal to generate a digital signal.

### Technical Solution

The representative configurations of the invention to achieve the above objects are described below.

According to one aspect of the invention, there is provided a method for monitoring biosignals using a wearable device, the method comprising the steps of: acquiring information on a result of performing a primary analysis of a biosignal measured by the device, using a primary analysis model trained to perform a primary analysis for detecting abnormal events from biosignals, and acquiring, from the biosignal, a partial biosignal associated with the result of performing the primary analysis; and performing a secondary analysis of the partial biosignal with reference to the information on the result of performing the primary analysis, using a secondary analysis model trained to perform a secondary analysis for detecting abnormal events from biosignals, wherein the primary analysis model is a relatively light-weighted model compared to the secondary analysis model.

According to another aspect of the invention, there is provided a method for monitoring biosignals using a wearable device, the method comprising the steps of: performing a primary analysis of a biosignal measured by the device, using a primary analysis model trained to perform a primary analysis for detecting abnormal events from biosignals; extracting, from the biosignal, a partial biosignal associated with a result of performing the primary analysis; and transmitting information on the result of performing the primary analysis and the partial biosignal to a server, wherein the server includes a secondary analysis model trained to perform a secondary analysis for detecting abnormal events from biosignals, and wherein the primary analysis model is a relatively light-weighted model compared to the secondary analysis model.

According to yet another aspect of the invention, there is provided a server for monitoring biosignals using a wearable device, the server comprising: a primary analysis result acquisition unit configured to acquire information on a result of performing a primary analysis of a biosignal measured by the device, using a primary analysis model trained to perform a primary analysis for detecting abnormal events from biosignals, and acquire, from the biosignal, a partial biosignal associated with the result of performing the primary analysis; and a secondary analysis unit configured to perform a secondary analysis of the partial biosignal with reference to the information on the result of performing the primary analysis, using a secondary analysis model trained to perform a secondary analysis for detecting abnormal events from biosignals, wherein the primary analysis model is a relatively light-weighted model compared to the secondary analysis model.

According to still another aspect of the invention, there is provided a device for monitoring biosignals using a wearable device, the device comprising: a primary analysis unit configured to perform a primary analysis of a biosignal measured by the device, using a primary analysis model trained to perform a primary analysis for detecting abnormal events from biosignals; and a primary analysis result management unit configured to extract, from the biosignal, a partial biosignal associated with a result of performing the primary analysis, and transmit information on the result of performing the primary analysis and the partial biosignal to a server, wherein the server includes a secondary analysis model trained to perform a secondary analysis for detecting abnormal events from biosignals, and wherein the primary analysis model is a relatively light-weighted model compared to the secondary analysis model.

In addition, there are further provided other methods, servers, and devices to implement the invention, as well as non-transitory computer-readable recording media having stored thereon computer programs for executing the methods.

### Advantageous Effects

According to the invention, it is possible to accurately monitor abnormal events from biosignals in real time using both a light-weighted artificial intelligence-based analysis model suitable for operation in a wearable device and an advanced artificial intelligence-based analysis model suitable for operation in a server.

According to the invention, it is possible to reduce the data size of a biosignal while ensuring high quality of the biosignal, so that the burden of communication between a wearable device and a server may be lowered and the amount of data that an analysis model should process to monitor biosignals may be reduced, which may contribute to light-weighting the entire process of monitoring the biosignals.

### BRIEF DESCRIPTION OF THE DRAWINGS

FIG. 1 schematically shows the configuration of an entire system for monitoring biosignals using a wearable device according to one embodiment of the invention.

FIG. 2 specifically shows the internal configuration of a server according to one embodiment of the invention.

FIG. 3 specifically shows the internal configuration of a device according to one embodiment of the invention.

FIG. 4 illustratively shows how to remove low-frequency noise from a biosignal measured by a wearable device according to one embodiment of the invention.

FIG. 5 illustratively shows how to extract, from a biosignal measured by a wearable device, a partial biosignal to be transmitted to a server according to one embodiment of the invention.

### DESCRIPTION OF THE REFERENCE NUMERALS

100: communication network

200: server

210: primary analysis result acquisition unit

220: secondary analysis unit

230: analysis model management unit

240: communication unit

250: control unit

300: device

310: primary analysis unit

320: primary analysis result management unit

330: communication unit

340: control unit

### BEST MODE FOR CARRYING OUT THE INVENTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

The biosignals to be monitored herein may encompass any type of biosignal that may be measured through a sensor provided in or capable of communicating with the device 300, and may include, for example, an electrocardiogram, a heart rate, brainwaves, and pulse. Meanwhile, it is noted that the biosignals according to the invention are not necessarily limited to those listed above, and may be expanded or changed without limitation as long as the objects of the invention may be achieved.

Further, the abnormal events that may be detected from the biosignals herein may include various cardiac disorder events associated with arrhythmia, such as premature atrial complex, premature ventricular complex, atrial fibrillation, atrial flutter, multifocal atrial tachycardia, paroxysmal supraventricular tachycardia, Wolf-Parkinson-White syndrome, ventricular tachycardia, ventricular fibrillation, and atrioventricular (AV) block. Meanwhile, it is noted that the abnormal events according to one embodiment of the invention are not necessarily limited to the cardiac disorder events listed above, and may be expanded or changed to various abnormal events associated with other organs (e.g., brain) or other body tissues (e.g., muscles).

Configuration of the Entire System

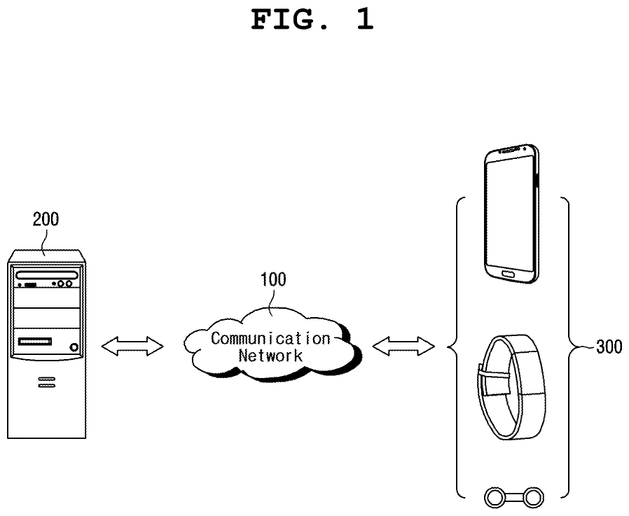

FIG. 1 schematically shows the configuration of the entire system for monitoring biosignals using a wearable device according to one embodiment of the invention.

As shown in FIG. 1, the entire system according to one embodiment of the invention may comprise a communication network 100, a server 200, and a device 300.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as radio frequency (RF) communication, WiFi communication, cellular communication (e.g., Long Term Evolution (LTE) or 5G communication), Bluetooth communication (more specifically, Bluetooth Low Energy (BLE) communication), infrared communication, and ultrasonic communication.

Next, the server 200 according to one embodiment of the invention may be a digital device having a memory means and a microprocessor for computing capabilities. The server 200 may be a typical server system.

The server 200 according to one embodiment of the invention may communicate with the device 300 to be described below via the communication network 100, and may function to: acquire information on a result of performing a primary analysis of a biosignal measured by the device, using a primary analysis model trained to perform a primary analysis for detecting abnormal events from biosignals, and acquire, from the biosignal, a partial biosignal associated with the result of performing the primary analysis; and perform a secondary analysis of the partial biosignal with reference to the information on the result of performing the primary analysis, using a secondary analysis model trained to perform a secondary analysis for detecting abnormal events from biosignals, thereby accurately monitoring abnormal events from biosignals in real time using artificial intelligence-based analysis models and a wearable device. Here, according to one embodiment of the invention, the primary analysis model is a relatively light-weighted model compared to the secondary analysis model.

The configuration and functions of the server 200 according to the invention will be discussed in more detail below. Meanwhile, although the server 200 has been described as above, the above description is illustrative, and it will be apparent to those skilled in the art that at least a part of the functions or components required for the server 200 may be implemented or included in the device 300 to be described below or an external system (not shown), as necessary.

Next, according to one embodiment of the invention, the device 300 is digital equipment that may function to connect to and then communicate with the server 200 via the communication network 100, and any type of portable digital equipment having a memory means and a microprocessor for computing capabilities, such as a smart phone and a tablet PC, may be adopted as the device 300 according to the invention. Further, according to one embodiment of the invention, the device 300 may include biosignal measurement sensors for acquiring various biosignals from a user's body (e.g., an electrocardiogram sensor, an electromyogram sensor, a heart rate sensor, a brainwave sensor, and a pulse sensor).

In particular, throughout the entire disclosure, the device 300 according to one embodiment of the invention should be understood as encompassing a wearable device (e.g., a smart watch or a smart patch) that may be constantly attached to the user's body to measure biosignals).

The device 300 according to one embodiment of the invention may communicate with the server 200 via the communication network 100, and may function to: perform a primary analysis of a biosignal measured by the device 300, using a primary analysis model trained to perform a primary analysis for detecting abnormal events from biosignals; extract, from the biosignal, a partial biosignal associated with a result of performing the primary analysis; and transmit information on the result of performing the primary analysis and the partial biosignal to the server, thereby accurately monitoring abnormal events from biosignals in real time using artificial intelligence-based analysis models and a wearable device.

Meanwhile, according to one embodiment of the invention, the server 200 or the device 300 may include an application (not shown) for supporting the functions necessary for monitoring biosignals using a wearable device. The application may be downloaded from an external application distribution server (not shown). Here, at least a part of the application may be replaced with a hardware device or a firmware device that may perform a substantially equal or equivalent function, as necessary.

Configuration of the Server

Hereinafter, the internal configuration of the server 200 crucial for implementing the invention and the functions of the respective components thereof will be discussed.

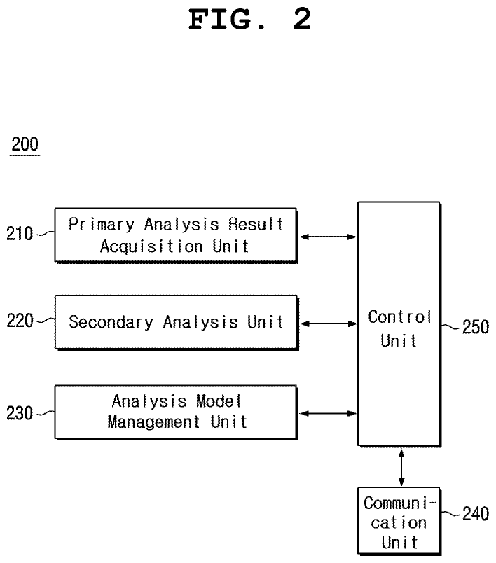

FIG. 2 specifically shows the internal configuration of the server 200 according to one embodiment of the invention.

As shown in FIG. 2, the server 200 according to one embodiment of the invention may comprise a primary analysis result acquisition unit 210, a secondary analysis unit 220, an analysis model management unit 230, a communication unit 240, and a control unit 250. According to one embodiment of the invention, at least some of the primary analysis result acquisition unit 210, the secondary analysis unit 220, the analysis model management unit 230, the communication unit 240, and the control unit 250 may be program modules that communicate with an external system. The program modules may be included in the server 200 in the form of operating systems, application program modules, or other program modules, while they may be physically stored in a variety of commonly known storage devices. Further, the program modules may also be stored in a remote storage device that may communicate with the server 200. Meanwhile, such program modules may include, but are not limited to, routines, subroutines, programs, objects, components, and data structures for performing specific tasks or executing specific abstract data types according to the invention as will be described below.

Meanwhile, although the server 200 has been described as above, the above description is illustrative, and it will be apparent to those skilled in the art that at least a part of the components or functions of the server 200 may be implemented or included in an external system (not shown), as necessary.

First, the primary analysis result acquisition unit 210 according to one embodiment of the invention may acquire information on a result of performing a primary analysis of a biosignal measured by the device 300, using a primary analysis model trained to perform a primary analysis for detecting abnormal events from biosignals. Further, the primary analysis result acquisition unit 210 according to one embodiment of the invention may acquire, from the biosignal measured by the device 300, a partial biosignal associated with the result of performing the primary analysis.

Next, the secondary analysis unit 220 according to one embodiment of the invention may perform a secondary analysis of the partial biosignal with reference to the information on the result of performing the primary analysis, using a secondary analysis model trained to perform a secondary analysis for detecting abnormal events from biosignals.

According to one embodiment of the invention, the primary analysis model provided in the device 300 may be a relatively light-weighted model compared to the secondary analysis model provided in the server 200. Further, according to one embodiment of the invention, the primary analysis model provided in the device 300 may be generated and distributed by the server 200.

Specifically, according to one embodiment of the invention, the primary analysis model provided in the device 300 is an analysis model trained to detect an abnormal event from the biosignal measured by the device 300, and may be a light-weighted analysis model that requires relatively less computing resources compared to the secondary analysis model provided in the server 200. For example, the primary analysis model may perform an analysis for determining only whether a cardiac disorder event has occurred from the biosignal.

Further, according to one embodiment of the invention, the secondary analysis model provided in the server 200 is an analysis model trained to detect an abnormal event from the partial biosignal transmitted from the device 300, and may be an advanced analysis model that requires relatively more computing resources compared to the primary analysis model provided in the device 300. For example, the secondary analysis model may perform an analysis for discriminating which type of cardiac disorder event specifically corresponds to the cardiac disorder event detected by the primary analysis model.

More specifically, according to one embodiment of the invention, at least one of a probability, vector, matrix, logic, and coordinate associated with a cardiac disorder may be outputted from the primary analysis model or the secondary analysis model, and the output may be classified or grouped into a specific cardiac disorder event (e.g., normal or abnormal) according to a certain criterion. (The grouping may be performed on the basis of a distance (e.g., K-means), a density (e.g., DB-SCAN), or the like.) In addition, the criterion may be predetermined or dynamically updated while the training is performed.

For example, the analysis model according to one embodiment of the invention may comprise an input layer, a hidden layer, and an output layer based on an artificial neural network. According to one embodiment of the invention, the analysis model may include an autoencoder, a generative adversarial net (GAN), a U-NET, and the like. Meanwhile, the analysis model according to the invention is not necessarily limited only to the learning models listed above, and may be changed to various learning models involved in supervised learning (in this case, labels for data may be further provided in the process of the learning), unsupervised learning, or reinforcement learning as long as the objects of the invention may be achieved.

Next, the analysis model management unit 230 according to one embodiment of the invention may generate an primary analysis model that is light-weighted to a level suitable for real-time operation in the device 300, and distribute the primary analysis model to the device 300. Further, the analysis model management unit 230 according to one embodiment of the invention may generate a secondary analysis model that is advanced to a level suitable for precise biosignal analysis in the server 200, and provide the secondary analysis model to the server 200.

More specifically, the analysis model management unit 230 according to one embodiment of the invention may generate an analysis model trained to detect abnormal events from biosignals, and light-weight the generated model using an artificial neural network model light-weighting algorithm such as pruning, quantization, and knowledge distillation. Further, the analysis model management unit 230 according to one embodiment of the invention may distribute the light-weighted model to the device 300 as a primary analysis model, so that a primary analysis may be performed in the device 300 having less computing resources compared to the server 200. However, the light-weighting algorithm according to one embodiment of the invention is not limited to those listed above, and may be diversely changed as long as the objects of the invention may be achieved.

Next, the communication unit 240 according to one embodiment of the invention may function to enable data transmission/reception from/to the primary analysis result acquisition unit 210, the secondary analysis unit 220, and the analysis model management unit 230.

Lastly, the control unit 250 according to one embodiment of the invention may function to control data flow among the primary analysis result acquisition unit 210, the secondary analysis unit 220, the analysis model management unit 230, and the communication unit 240. That is, the control unit 250 according to the invention may control data flow into/out of the server 200 or data flow among the respective components of the server 200, such that the primary analysis result acquisition unit 210, the secondary analysis unit 220, the analysis model management unit 230, and the communication unit 240 may carry out their particular functions, respectively.

Configuration of the Device

Hereinafter, the internal configuration of the device 300 crucial for implementing the invention and the functions of the respective components thereof will be discussed.

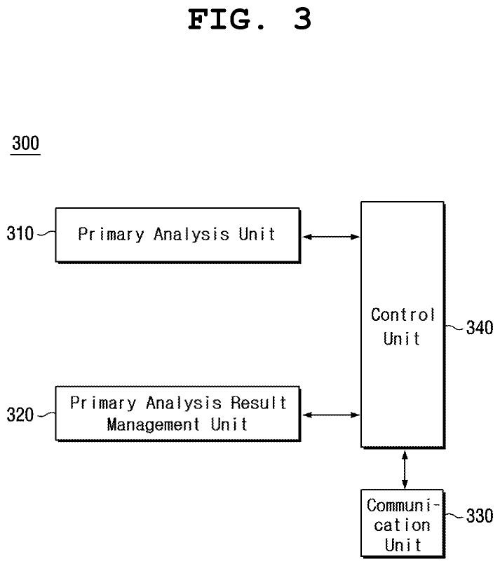

FIG. 3 specifically shows the internal configuration of the device 300 according to one embodiment of the invention.

As shown in FIG. 3, the device 300 according to one embodiment of the invention may comprise a primary analysis unit 310, a primary analysis result management unit 320, a communication unit 330, and a control unit 340. According to one embodiment of the invention, at least some of the primary analysis unit 310, the primary analysis result management unit 320, the communication unit 330, and the control unit 340 may be program modules that communicate with an external system. The program modules may be included in the device 300 in the form of operating systems, application program modules, or other program modules, while they may be physically stored in a variety of commonly known storage devices. Further, the program modules may also be stored in a remote storage device that may communicate with the device 300. Meanwhile, such program modules may include, but are not limited to, routines, subroutines, programs, objects, components, and data structures for performing specific tasks or executing specific abstract data types according to the invention as will be described below.

Meanwhile, although the device 300 has been described as above, the above description is illustrative, and it will be apparent to those skilled in the art that at least a part of the components or functions of the device 300 may be implemented or included in an external system (not shown), as necessary.

First, the primary analysis unit 310 according to one embodiment of the invention may perform a primary analysis of a biosignal measured by the device 300, using a primary analysis model trained to perform a primary analysis for detecting abnormal events from biosignals.

Next, the primary analysis result management unit 320 according to one embodiment of the invention may extract, from the biosignal measured by the device 300, a partial biosignal associated with a result of performing the primary analysis.

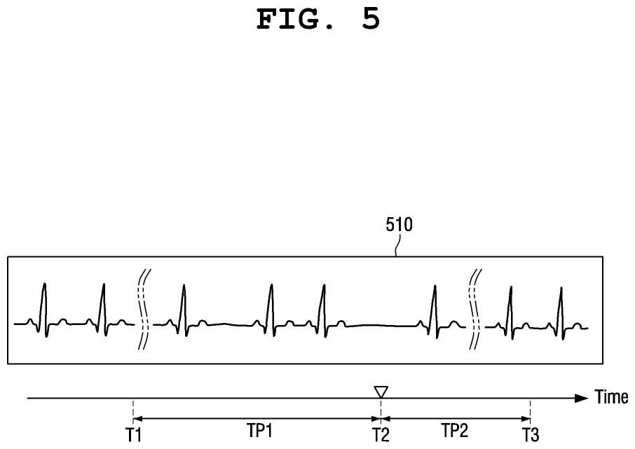

FIG. 5 illustratively shows how to extract, from a biosignal measured by a wearable device, a partial biosignal to be transmitted to a server according to one embodiment of the invention.

Referring to FIG. 5, when a partial biosignal to be transmitted to the server 300 for a secondary analysis is extracted from a biosignal 510 measured by the device 300, a time period of the partial biosignal may be specified with respect to a time point T2 at which an abnormal event is determined to have occurred according to a result of performing a primary analysis. For example, the time period of the partial biosignal may be specified to include a time period TP1 temporally preceding T2 and a time period TP2 temporally following T2.

Further, the primary analysis result management unit 320 according to one embodiment of the invention may transmit information on the result of performing the primary analysis and the extracted partial biosignal to the server 200. As described above, the server 200 according to one embodiment of the invention may perform a secondary analysis for the abnormal event using the information on the result of performing the primary analysis and the partial biosignal transmitted from the device 300 as above.

Meanwhile, a data light-weighting unit (not shown) according to one embodiment of the invention may light-weight data corresponding to the biosignal measured by the device 300, by removing low-frequency noise from the biosignal and adjusting the number of bits of data extracted (or sampled) from the biosignal (i.e., data bit reduction), in the process of performing analog-to-digital conversion on the biosignal.

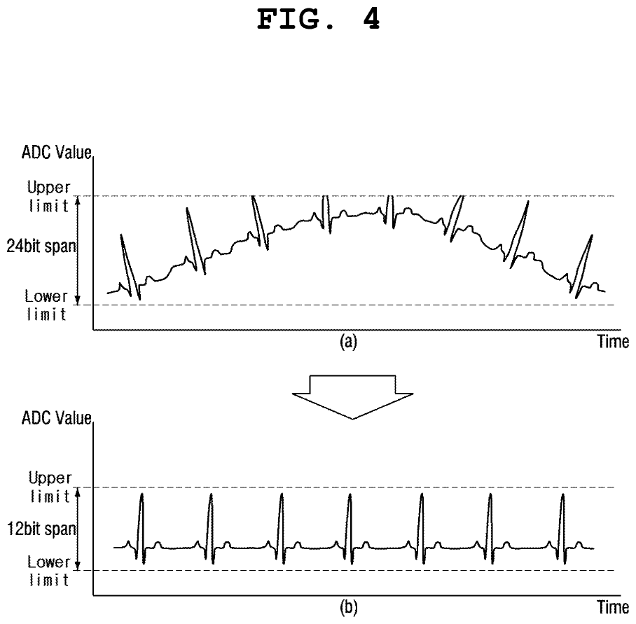

FIG. 4 illustratively shows how to remove low-frequency noise from a biosignal measured by a wearable device according to one embodiment of the invention.

Referring to FIG. 4, the data light-weighting unit according to one embodiment of the invention may remove low-frequency noise from a biosignal measured by the device 300. According to one embodiment of the invention, the range in which the signal values of the biosignal are distributed may be narrowed as the low-frequency noise is removed from the biosignal. Further, the data light-weighting unit according to one embodiment of the invention may generate a digital signal by extracting (or sampling), from the analog signal whose distribution range of the signal values is narrowed as the low-frequency noise is removed as above, data with the number of bits corresponding to the range that may cover the signal values.

For example, before the low-frequency noise is removed from the biosignal, it is difficult to sufficiently cover the signal values of the biosignal even if data is extracted with 24 bits (see (a) of FIG. 5). However, after the low-frequency noise is removed from the biosignal according to the invention, it can be seen that the signal values of the biosignal may be sufficiently covered by only extracting data with 12 bits, which is only half of 24 bits (see (b) of FIG. 5).

As described above, according to the invention, it is possible to reduce the data size of a biosignal while ensuring high quality of the biosignal. Therefore, it is possible to lower the burden of communication between the wearable device and the server, and reduce the amount of data that an analysis model should process to monitor biosignals, which may contribute to light-weighting the entire process of monitoring the biosignals.

Next, the communication unit 330 according to one embodiment of the invention may function to enable data transmission/reception from/to the primary analysis unit 310 and the primary analysis result management unit 320.

Lastly, the control unit 340 according to one embodiment of the invention may function to control data flow among the primary analysis unit 310, the primary analysis result management unit 320, and the communication unit 330. That is, the control unit 340 according to the invention may control data flow into/out of the device 300 or data flow among the respective components of the device 300, such that the primary analysis unit 310, the primary analysis result management unit 320, and the communication unit 330 may carry out their particular functions, respectively.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

1. A method for monitoring biosignals using a wearable device, the method comprising the steps of:
acquiring information on a result of performing a primary analysis of a biosignal measured by the device, using a primary analysis model trained to perform a primary analysis for detecting abnormal events from biosignals, and acquiring, from the biosignal, a partial biosignal associated with the result of performing the primary analysis; and
performing a secondary analysis of the partial biosignal with reference to the information on the result of performing the primary analysis, using a secondary analysis model trained to perform a secondary analysis for detecting abnormal events from biosignals,
wherein the primary analysis model is a relatively light-weighted model compared to the secondary analysis model.

2. A method for monitoring biosignals using a wearable device, the method comprising the steps of:
performing a primary analysis of a biosignal measured by the device, using a primary analysis model trained to perform a primary analysis for detecting abnormal events from biosignals;
extracting, from the biosignal, a partial biosignal associated with a result of performing the primary analysis; and
transmitting information on the result of performing the primary analysis and the partial biosignal to a server,
wherein the server includes a secondary analysis model trained to perform a secondary analysis for detecting abnormal events from biosignals, and
wherein the primary analysis model is a relatively light-weighted model compared to the secondary analysis model.

3. The method of claim 2, wherein at least one filter removes low-frequency noise from the biosignal measured by the device, and
wherein analog-to-digital conversion is performed on the biosignal from which the low-frequency noise is removed, such that a number of bits of data extracted from an analog signal to generate a digital signal is determined within a range capable of covering signal values of the biosignal from which the low-frequency noise is removed.

4. The method of claim 2, wherein a time period of the partial biosignal is specified with respect to a time point at which an abnormal event is determined to have occurred according to the result of performing the primary analysis.

5. A non-transitory computer-readable recording medium having stored thereon a computer program for executing the method of claim 1.

6. A server for monitoring biosignals using a wearable device, the server comprising:
a primary analysis result acquisition unit configured to acquire information on a result of performing a primary analysis of a biosignal measured by the device, using a primary analysis model trained to perform a primary analysis for detecting abnormal events from biosignals, and acquire, from the biosignal, a partial biosignal associated with the result of performing the primary analysis; and
a secondary analysis unit configured to perform a secondary analysis of the partial biosignal with reference to the information on the result of performing the primary analysis, using a secondary analysis model trained to perform a secondary analysis for detecting abnormal events from biosignals,
wherein the primary analysis model is a relatively light-weighted model compared to the secondary analysis model.

7. A device for monitoring biosignals using a wearable device, the device comprising:
a primary analysis unit configured to perform a primary analysis of a biosignal measured by the device, using a primary analysis model trained to perform a primary analysis for detecting abnormal events from biosignals; and
a primary analysis result management unit configured to extract, from the biosignal, a partial biosignal associated with a result of performing the primary analysis, and transmit information on the result of performing the primary analysis and the partial biosignal to a server,
wherein the server includes a secondary analysis model trained to perform a secondary analysis for detecting abnormal events from biosignals, and
wherein the primary analysis model is a relatively light-weighted model compared to the secondary analysis model.

8. The device of claim 7, wherein at least one filter removes low-frequency noise from the biosignal measured by the device, and
wherein analog-to-digital conversion is performed on the biosignal from which the low-frequency noise is removed, such that a number of bits of data extracted from an analog signal to generate a digital signal is determined within a range capable of covering signal values of the biosignal from which the low-frequency noise is removed.

9. The device of claim 7, wherein a time period of the partial biosignal is specified with respect to a time point at which an abnormal event is determined to have occurred according to the result of performing the primary analysis.

10. A non-transitory computer-readable recording medium having stored thereon a computer program for executing the method of claim 2.
